(19) United States
(12) Patent Application Publication
(10) Pub. No.: US 2017/0332938 A1
(43) Pub. Date: Nov. 23, 2017

(54) METHODS FOR CO-IMAGING TISSUE STIFFNESS AND BLOOD FLOW IN AN MRI SCAN

(71) Applicant: Ohio State Innovation Foundation, Columbus, OH (US)

(72) Inventors: Rizwan Ahmad, Hilliard, OH (US); Arunark Kolipaka, Delaware, OH (US)

(21) Appl. No.: 15/598,661

(22) Filed: May 18, 2017

Related U.S. Application Data
(60) Provisional application No. 62/338,729, filed on May 19, 2016.

(51) Int. Cl. A61B 5/055; A61B 5/00; G01R 33/563

(52) U.S. Cl. CPC A61B 5/055; G01R 33/56358; A61B 5/0048; G01R 33/56316

(57) ABSTRACT

Techniques for co-imaging tissue stiffness and blood flow using a single MRI scan are disclosed. The methods use a combined gradient waveform that provides adequate sensitivity for concurrent encodings of flow and tissue stiffness. During a scan, the application of the combined gradient waveform, in the presence of an applied oscillatory motion, simultaneously encodes both flow and stiffness information into the phase of the resulting MRI image. To separate the flow information from the tissue displacement caused by the oscillatory motion, a Fourier transform applied along the direction of applied oscillatory motion. After the transformation, baseband information (flow velocity) may be separated from modulated information (tissue displacement). The separated data may be used to create a velocity map and a displacement map, which can then be converted to a stiffness map.

### CROSS-REFERENCE TO RELATED APPLICATION

This non-provisional application claims the benefit of U.S. Provisional Application No. 62/338,729, filed May 19, 2016, the whole disclosure of which is incorporated by reference herein.

### GOVERNMENT SUPPORT

The present application was made with government support under R01HL24096 awarded by the National Institute of Health and under 13SDG14690027 awarded by the American Heart Association. The government has certain rights in the invention.

### FIELD OF THE INVENTION

The present invention relates to magnetic resonance imaging (MRI) and more specifically to techniques for imaging tissue stiffness and blood flow simultaneously.

### BACKGROUND

Changes in hemodynamics (e.g., blood flow) and viscoelasticity (e.g., tissue stiffness) have been linked to a wide range of cardiovascular conditions, and the measurement of each may provide complementary information that can elucidate complex disease mechanisms and/or improve diagnosis. For example, in patients with aortic aneurysm or valvular disease, hemodynamics may quantify a wall shear stress exerted by blood on a diseased section of the organ, and tissue stiffness may provide the ability of the organ to comply with the wall shear stress. As a result, the collective information from hemodynamics and tissue stiffness can potentially yield a more comprehensive biomarker for diagnosis and prognosis of complex diseases.

While phase-contrast magnetic resonance imaging (PC-MRI) has been used to examine blood flow and tissue stiffness, it is generally not feasible, in clinical settings, to image both using a single scan acquisition for a variety of reasons. First, the acquisition time (i.e., scan time) necessary to acquire two datasets, one for blood-flow imaging and one for tissue-stiffness imaging, is extremely long. Second, patients are typically unable to sustain the multiple, long breath-holds necessary for two acquisitions. Third, variation in breath-holds from one scan to another may cause a registration mismatch between the resulting images. This variation between datasets makes interpretation difficult, which may lead to a misdiagnosis.

A need, therefore, exists for acquisition and processing methods to image blood flow and tissue stiffness from a single dataset (i.e., from a single acquisition). Use of these methods may offer a plurality of advantages, including, but not limited to (i) reduced scan time, (ii) automatic registration of datasets, (iii) reduced susceptibility to physiological changes, (iv) eased breath-hold requirements, and (v) complementary information for improved understanding/diagnosis.

### SUMMARY

Accordingly, a first data acquisition method disclosed herein embraces encoding both tissue motion and fluid flow in a magnetic resonance imaging (MRI) acquisition. For this simultaneous encoding, a combined gradient (CG) waveform is created that is a weighted combination of a motion encoding gradient (MEG) waveform and a velocity encoding gradient (VEG) waveform. During the MRI acquisition, an oscillatory motion is applied to a tissue of interest located within the MRI field of view. The oscillatory motion creates shear waves in the tissue. Also during the MRI acquisition, the CG waveform is applied to one or more gradients in a pulse sequence. The MEG component of the CG waveform encodes the shear waves into the phase of the spins in the tissue, while the VEG component of the CG waveform encodes the fluid flow into the phase of spins in the tissue.

In accordance with an aspect of the first method, the pulse sequence may be a spin-echo (SE) or a gradient recalled echo (GRE) pulse sequence, and in these cases the CG waveform is be applied to one or more gradients after a 90 degree radio frequency (RF) pulse and before a readout gradient.

In accordance with another aspect of the first method, the one or more gradients may include the slice-select gradient, the frequency encode gradient, and the phase-encode gradient, and the one or more gradients may be aligned with the direction of the shear wave's propagation and/or the direction of the fluid flow.

In accordance with another aspect of the first method, the MEG waveform, such as a W₁₋₂₋₁ waveform used in magnetic resonance elastography (MRE), has a nonzero inner product with the gradient waveform and the oscillation. In some cases, the W₁₋₂₋₁ waveform may be adjusted to match the frequency of the oscillatory motion. In other cases, the W₁₋₂₋₁ waveform may be adjusted to mismatch the frequency of the oscillatory motion. In still other cases, the oscillatory motion may be adjusted to have a particular phase offset with the W₁₋₂₋₁ waveform.

In accordance with another aspect of the first method, the VEG waveform, such as a W₁₋₁ waveform used in magnetic resonance velocity imaging, has a non-zero first moment.

In accordance with another aspect of the first method, the application of the CG waveform to a gradient during a pulse sequence for an MRI acquisition creates an accumulation of phase in the spins that corresponds to both (i) tissue displacement caused by the shear waves and (ii) fluid-flow velocity.

In accordance with another aspect of the first method, the CG waveform may be calculated using the equation

CG=(1−k₁)×W₁₋₂₋₁+k₁×W₁₋₁,

wherein CG is the combined waveform, W₁₋₂₋₁ is a repeating-bipolar waveform used in magnetic resonance elastography (MRE), W₁₋₁ is a nonrepeating-bipolar waveform used in magnetic resonance velocity imaging, and k₁ is a constant that is adjustable from zero to one. In some cases, k₁ may be adjusted so, during the MRI acquisition, the accumulation of the spin phase resulting from tissue displacement and the accumulation of spin phase resulting from fluid-flow velocity are approximately equal.

In general, W₁₋₂₋₁ may be a waveform in which the inner product between the waveform and the sinusoidal oscillation of spins in space is large in comparison to the waveform's first moment. Additionally, W₁₋₁ may a waveform in which the inner product between the waveform and the sinusoidal oscillation of spins in space is small in comparison to the waveform's first moment.

Additionally, the first method may further include the steps of (i) phase shifting the applied oscillatory motion to create a phase offset between the MEG waveform (e.g., the MEG component of the CG waveform) and the oscillatory motion and (ii) applying the CG waveform to one or more gradients in a pulse sequence for a subsequent MRI acquisition. Then, repeating these steps using different offsets to sample the shear waves as they propagate through the tissue.

The entire coding process may then repeated with the reverse polarity of CG (−1×CG) to create two datasets for each phase offset. Through this, the background phase may be diminished through a conjugate multiplication of the positive CG dataset and the negative CG dataset, and a phase map for each offset may be created.

The first method's simultaneous acquisition of a tissue stiffness map and a blood flow map from a single MRI scan requires less time than would be obtained by separately acquiring a tissue stiffness map from a first MRI scan and a blood flow map from a second MRI scan.

A second data processing method is also disclosed herein. The second method embraces extracting (i) a tissue stiffness map and (ii) a blood flow map, both from the phase maps extracted in the first method. The resulting phase images are then transformed using a Fourier transform applied along the offsets. The transformation decouples the information into baseband, first, and second harmonics of the frequency of the oscillatory motion. The flow-related phase energy is grouped around a baseband and the external oscillatory wave frequency related phase energy is grouped around the first harmonic. The first-harmonic information is used to create the map of tissue stiffness in the subject, while the baseband information is used to create the map of blood flow in the subject.

The foregoing illustrative summary, as well as other exemplary objectives and/or advantages, and the manner in which the same are accomplished, are further explained within the following detailed description and its accompanying drawings.

### BRIEF DESCRIPTION OF THE DRAWINGS

FIG. 1 graphically depicts an exemplary MRI pulse sequence in accordance with the present disclosure.

FIG. 2 graphically depicts an exemplary velocity encoding gradient (VEG) waveform in accordance with the present disclosure.

FIG. 3 graphically depicts an exemplary MRI pulse sequence with a velocity encoding gradient (VEG) waveform (W₁₋₁) applied to a gradient in accordance with the present disclosure.

FIG. 4 graphically depicts an exemplary motion encoding gradient (MEG) waveform in accordance with the present disclosure.

FIG. 5 graphically depicts an exemplary MRI pulse sequence with a motion encoding gradient (MEG) waveform (W₁₋₂₋₁) applied to a gradient in accordance with the present disclosure.

FIGS. 6A-6F graphically depict various combined gradient (CG) waveforms in accordance with the present disclosure.

FIG. 7 depicts a graph illustrating the phase accumulation for velocity and motion resulting from various weighted combinations of MEG and VEG.

FIG. 8 graphically depicts an exemplary MRI pulse sequence with a CG waveform applied to a gradient for combined velocity and MRE imaging.

FIG. 9 depicts a flowchart illustrating an exemplary method for simultaneously obtaining a tissue stiffness map and a blood flow map via an MRI in accordance with the present disclosure.

FIG. 10A graphically depicts phase images at different offsets exhibiting the propagation of a shear wave through the right femoral artery.

FIG. 10B graphically depicts a stiffness map corresponding to the phase images of FIG. 10A.

FIG. 11A graphically depicts a blood flow map.

FIG. 11B depicts a peak-velocity profile corresponding to the one-dimensional segment across the right femoral artery in FIG. 11A.

### DETAILED DESCRIPTION

MRE and velocity imaging are phase-contrast based techniques used in MRI to quantify tissue stiffness and flow velocity, respectively. Both techniques use special encoding waveforms applied to gradients during an MRI pulse sequence (e.g., spin echo, gradient recalled echo, etc.).

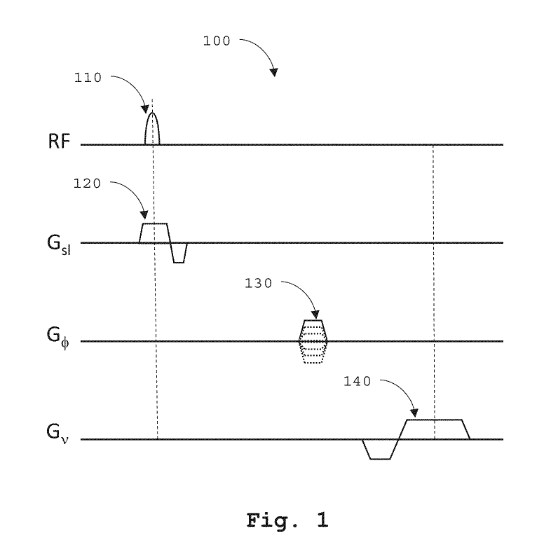

An exemplary gradient-recalled echo (GRE) pulse sequence 100 is shown in FIG. 1. A radio-frequency pulse (i.e., 90-degree RF pulse) 110 is applied to subject at the same time as a slice-select gradient (Gₛ₁) 120 in order to excite spins in a particular “slice” of the subject. The excited spins relax back to their resting state, and the energy released by the spins is detected and used to form images. As part of this process, the spins are encoded spatially using phase gradients (G_{φ}) 130 and a frequency (i.e., readout) gradient (Gᵥ) 140. During acquisition, the phase encoding gradient is iterated. As a result, the phase gradient shown in FIG. 1 has multiple amplitudes (shown in dotted lines). In the present disclosure, the term “gradients” generally refers to these gradients used for spatially encoding.

In velocity (i.e., flow) imaging, the spins are additionally phase encoded so that the spin phases also correspond to the velocity of fluid (e.g., blood, spinal fluid, etc.) flow. To achieve this, a velocity encoding gradient (VEG) waveform 200 is applied to one or more of the gradients during the pulse sequence. Typically, bipolar gradients are preferred for flow imaging due to their high first moment (M1). Phase accumulation occurs when a spin experiences a VEG gradient waveform with a non-zero (e.g., high) M1. Mathematically, the phase accumulation is given by:

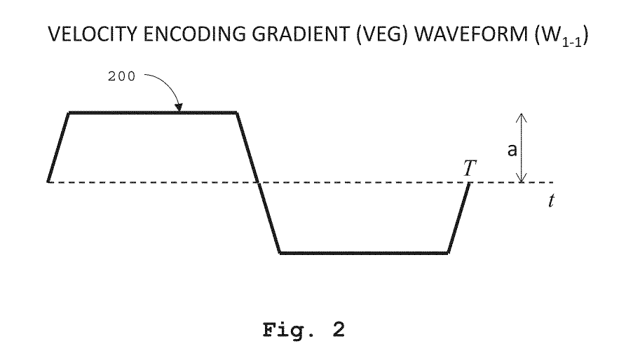

γv∫₀^{TE}Gt dt,

where γ is the gyromagnetic ratio, v is the velocity of the spin, t is time, ∫₀^{TE} Gt is first moment, and TE is the echo time (i.e., the time between the 90 degree RF pulse and readout). A typical VEG waveform 200 is shown in FIG. 2. The waveform is a (non-repeating) bipolar waveform with equal positive and negative portions. As a result, the waveform is referred to as “W₁₋₁”.

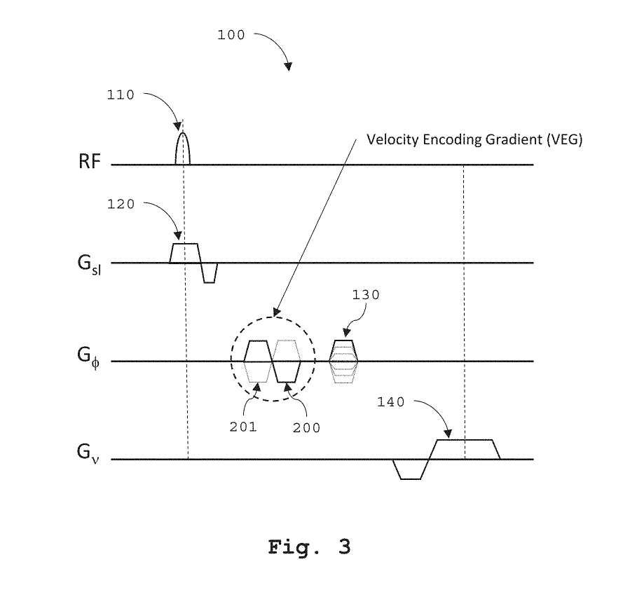

A GRE pulse sequence 100 with a VEG waveform 200 applied to a gradient (G_{φ}) is shown in FIG. 3. The VEG waveform 200 may be applied to encode spins along a particular physical direction that may or may not be aligned with the gradients. As a result, the VEG waveform 200 may be applied to the slice-select, the phase, or the frequency gradient. In addition, it is possible to apply the VEG waveform 200 to more than one gradient in order to encode spins in directions defined by the component directions of the gradients. As shown in FIG. 3, the VEG waveform 200 is applied to the phase encoding gradient (G_{φ}). To cancel background phase, the acquisition is repeated with VEG of positive and negative polarities, as shown by the dotted VEG waveform 201.

In MR elastography, the spins are phase encoded so that the spin phases correspond to the motion of tissue (i.e., shear waves) caused by applying a mechanical vibration to the subject during scan. To achieve this, a motion encoding gradient (MEG) waveform is applied to one or more of the gradients during the pulse sequence. Typically, repeating W₁₋₂₋₁ gradients are preferred for MRE imaging due to their low first moment (M1) and hence insensitivity to flow. Phase accumulation occurs when the MEG waveform is synchronized with the mechanical motion (i.e., oscillatory motion) applied to the tissues of a subject during the scan. Mathematically, the magnitude of phase accumulation is given by:

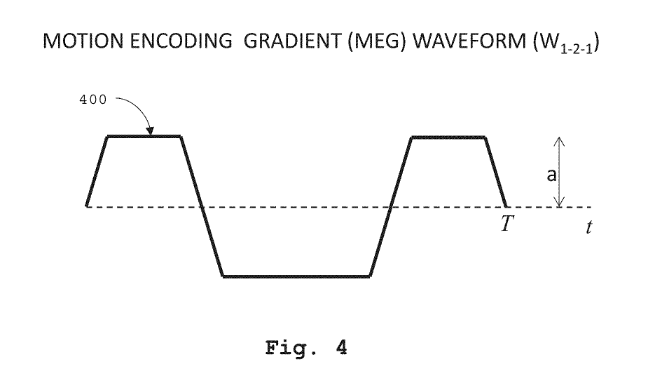

0.5γNT<G,ξ>,

where N is the number of gradient cycles, G is the encoding gradient waveform, ξ is the sinusoidal oscillation of spin in space, T is the period of waveform, and <, > is the inner product. A MEG waveform 400 is shown in FIG. 4. The waveform is a (repeating) waveform with negative portions having an area double that of either of the two positive portions. As a result, the waveform is referred to as “W₁₋₂₋₁”. Typically, W₁₋₂₋₁ (i.e., 1-2-1) waveforms are preferred for MRE because they provide high sensitivity to oscillatory motion and low sensitivity to blood flow that can otherwise overwhelm the MRE information. The flow sensitivity is directly proportional to the first moment, M1, of the gradient waveform and the 1-2-1 gradients offer small M1 values and thus are relatively insensitive to blood flow.

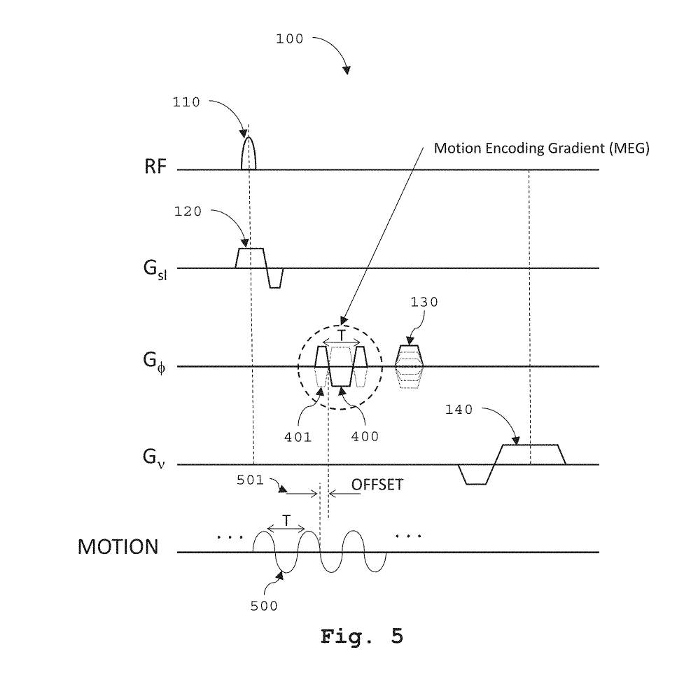

A GRE pulse sequence 100 with a MEG waveform 400 applied to a gradient (G_{φ}) is shown in FIG. 5. The MEG waveform 400 may be applied to encode spins along a particular physical direction that may or may not be aligned with the gradients. As a result, the MEG waveform may be applied to the slice-select gradient (Gₛ₁), the phase gradient (G_{φ}), or the frequency gradient (Gᵥ). In addition, it is possible to apply the MEG waveform to more than one gradient in order to encode spins in directions defined by the component directions of the gradients. As shown in FIG. 5, the MEG waveform 400 is applied along the phase encoding gradient (G_{φ}). The MEG waveform 400 and an oscillatory tissue motion 500 (resulting from an applied mechanical motion and shown in FIG. 5 as “MOTION”) are coherent (i.e., have matching frequencies and a fixed phase relationship). By adjusting the phase offset 501 between the motion and the MEG waveform, maps (i.e., snapshots) of the shear wave as it propagates through the tissue may be obtained during acquisition. In other words, by phase shifting the applied oscillatory motion, new phase offsets between the MEG waveform and the oscillatory motion may be created. Then, by imaging at different phase shifts, the shear waves may be sampled as they propagate through the tissues. To diminish (e.g., subtract, cancel, etc.) background phase, acquisition for each offset is repeated with a MEG of positive and negative polarities, as shown in FIG. 5 by the dotted MEG waveform 401. Using information resulting from the sampled shear wave propagation, tissue stiffness may be obtained. As a result, acquisitions for a number of different offsets may be required (e.g., sufficient to satisfy the Nyquist sampling theorem) to determine tissue stiffness (i.e., elasticity).

Typically, MRE and velocity imaging are performed as separate scans. The present disclosure embraces a co-imaging technique that utilizes a combined gradient (CG) waveform suitable for encoding both velocity and motion. The CG waveform offers a trade-off between <G, ξ> and ∫₀^{TE}Gt dt. The CG waveform may be customized to control the sensitivity (i.e., phase accumulation) of MRE-related oscillation and the velocity. As an example, varying k₁ in the CG waveform,

CG=(1−k₁)×W₁₋₂₋₁+k₁×W₁₋₁,

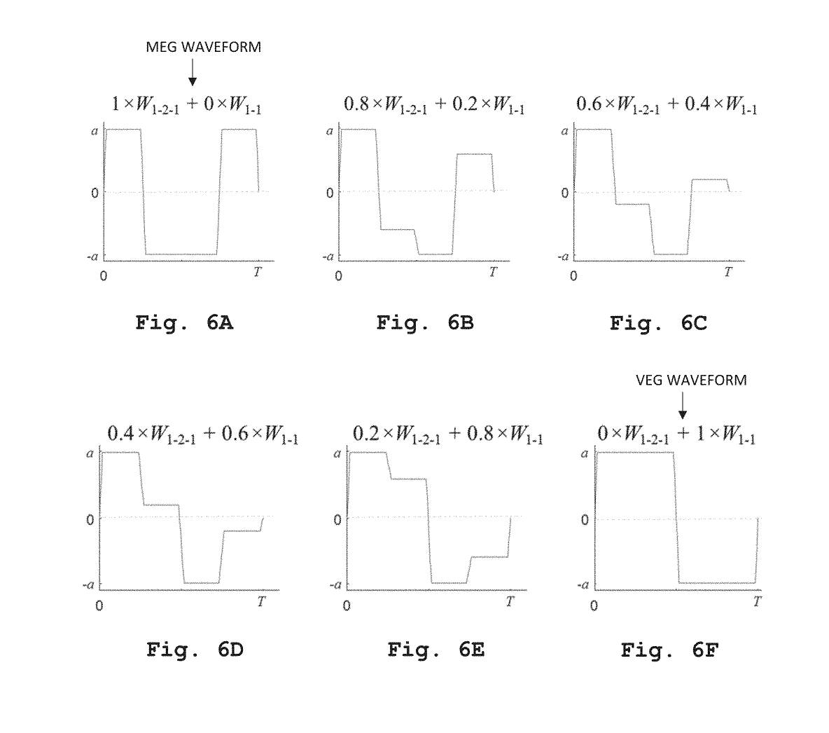

offers a sensitivity tradeoff. FIGS. 6A-6F illustrate various CG waveforms, each having different weighted combinations of W₁₋₂₋₁ and W₁₋₁ resulting from different k₁ values. Specifically, FIG. 6A illustrates a CG waveform for k₁=0 (i.e., a MEG waveform 400), FIG. 6B illustrates a CG waveform for k₁=0.2, FIG. 6C illustrates a CG waveform for k₁=0.4, FIG. 6D illustrates a CG waveform for k₁=0.6, FIG. 6E illustrates a CG waveform for k₁=0.8, and FIG. 6F illustrates a CG waveform for k₁=1 (i.e., a VEG waveform 200).

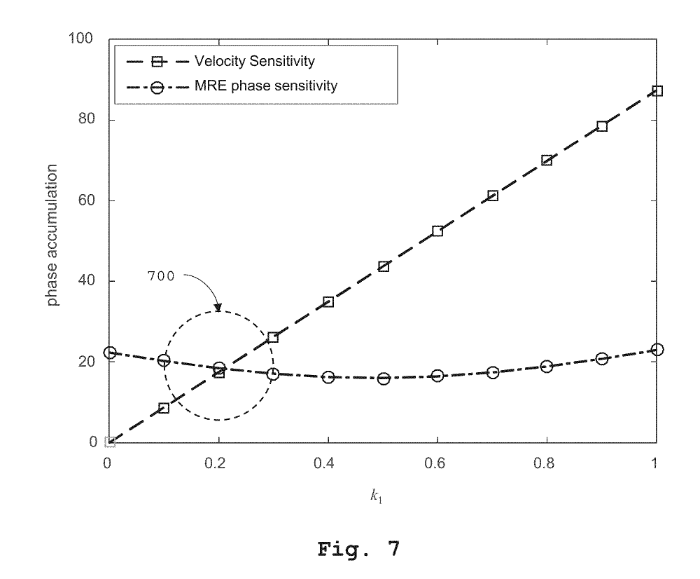

FIG. 7 illustrates (i.e., for the CG waveform: CG=(1−k₁)×W₁₋₂₋₁+k₁×W₁₋₁) the impact of 1>k₁>0 on phase accumulation due to MRE and flow. By adjusting k₁, the relative sensitivity to MRE-related phase and the velocity related phase can be controlled. For FIG. 7, phase accumulation is calculated for a single spin subjected to a linear gradient. For the MRE results, phase accumulation is given by 0.5γNT<G, ξ>, where γ is the gyromagnetic ratio, N is the number of gradient cycles, G is the encoding gradient waveform, ξ is the sinusoidal oscillation of spin in space, T is the period of waveform, and <, > represents the inner product. For the velocity encoding results, phase accumulation is given by γv∫₀^{TE}GTdt, where v is the velocity of the spin, t is time, and TE is the echo time. FIG. 7 results from a VEG/MEG amplitude (a) of 2.7 Gauss/centimeter, a velocity (v) of 50 centimeters/second, a mechanical motion amplitude M of 50 μm (i.e., microns), and VEG/MEG period (T) of 10 ms (i.e., milliseconds).

The choice of k₁ is made to insure that both phenomena can be detected in the resulting MRI images (e.g., phase images). As highlighted 700 in the exemplary graph shown in FIG. 7, the phase accumulation for velocity and MRE is equal at a k₁ of 0.2. For this example, selection of the k₁ around this point (e.g., illustrated by the dashed circle) would likely be acceptable. The ultimate choice of k₁ may be made using experience, test conditions, and/or a preliminary scan. It is envisioned that the selection process may be automated.

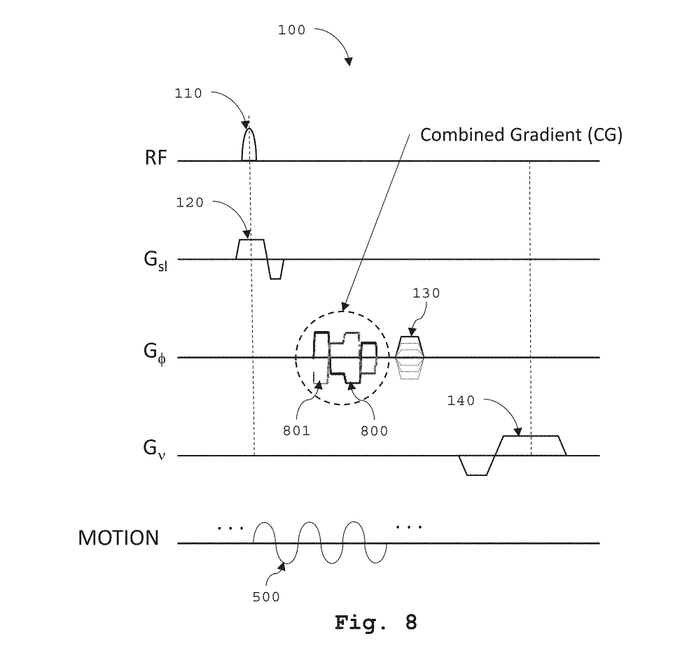

As shown in FIG. 8, a GRE pulse sequence 100 with a CG waveform (k₁=0.2) 800 (see FIG. 6B) can be applied to a gradient (G_{φ}). As previously described, the CG waveform 800 may be applied to any of the gradients and may be applied more than one gradient. Also as discussed, the polarity of the CG waveform 800 may be reversed during the acquisition to improve sensitivity to motion/velocity and to cancel background phase, as shown by the dotted CG waveform 801.

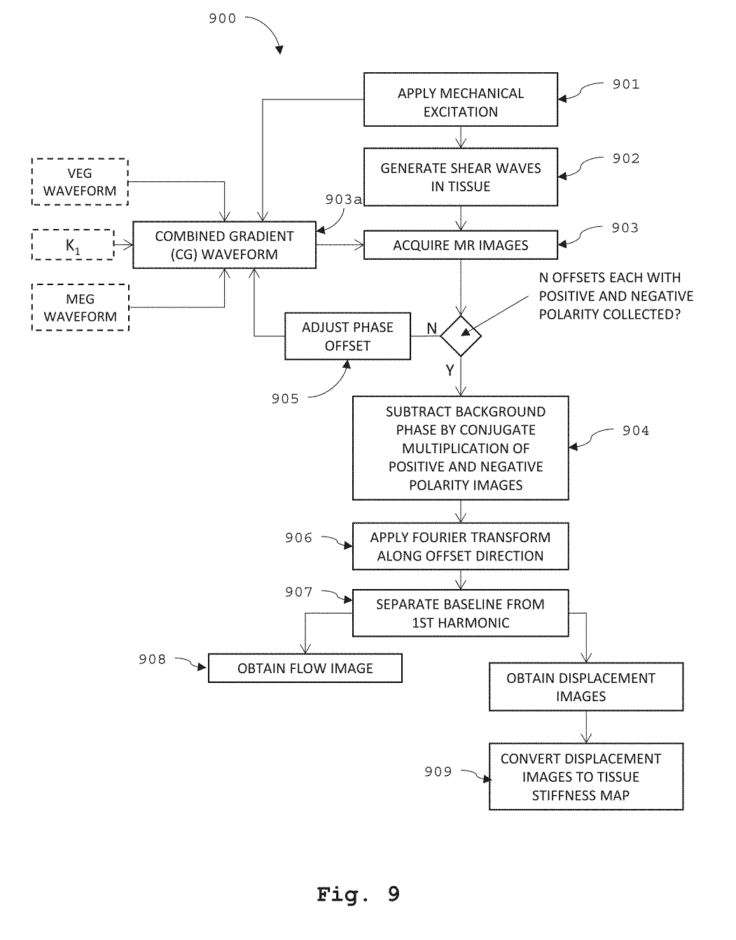

A co-imaging MRI acquisition method 900 is illustrated in the flow diagram shown in FIG. 9. The method obtains a pair of complex-valued images by applying an oscillatory (mechanical) motion 901 to a subject (i.e., tissues of the subject) to create shear waves in the tissues 902. MR images are acquired 903 using a first combined gradient waveform 903a applied to one or more gradients is performed to obtain a first complex-valued image (positive polarity image). Next, a second CG, which has a polarity opposite to the first CG is applied and a second complex-valued image (negative polarity image) is obtained to obtain an image pair comprised of the first and second complex-valued images. A phase map is created and the background phase is diminished by a conjugate multiplication of the positive and negative polarity images 904.

The phase offset between the CG waveform and the oscillatory motion may be repeatedly adjusted (e.g., for a number, N, of offsets) 905 to obtain additional phase maps for different phase offsets.

Data processing techniques are used to separate the phase contributions from MRE and velocity. To delineate phase encoded for MRE and velocity, a Fourier transform may be computed along the offset dimension 906 (i.e., along the temporal evaluation of the shear wave). The Fourier transform will result in signals generally organized into different harmonics.

The signal from MRE may be grouped about the first harmonic (i.e., 1x) of the oscillatory signal applied to the tissues of a subject; while the signal from flow may be grouped about the 0^{th} harmonic (i.e., baseband) since the phase due to flow is not modulated by an external mechanical stimulus. Because of the separation in frequency (i.e., Fourier domain), the two signals may be separated (e.g., by filtering) 907. After separating the two signals, the two resulting signals can be processed using known MRI methods to obtain a flow image 908 and a tissue stiffness map 909.

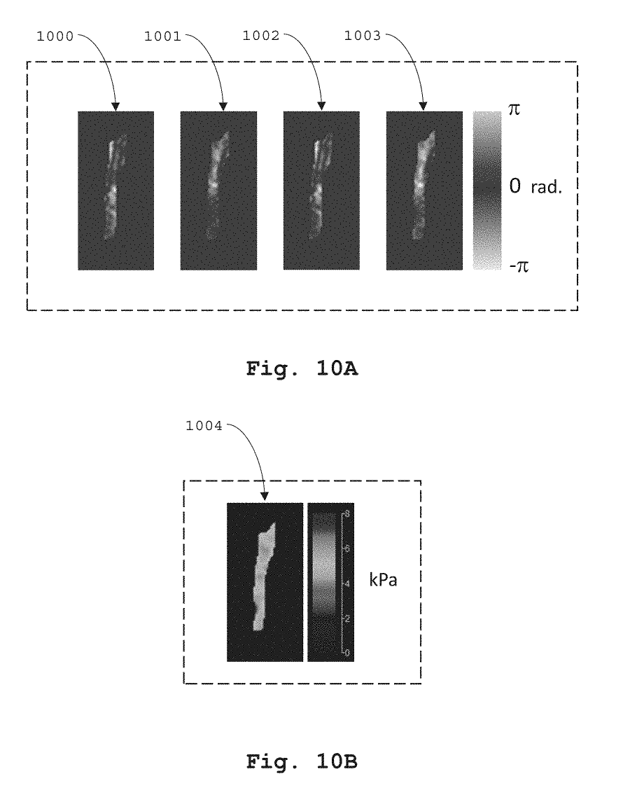

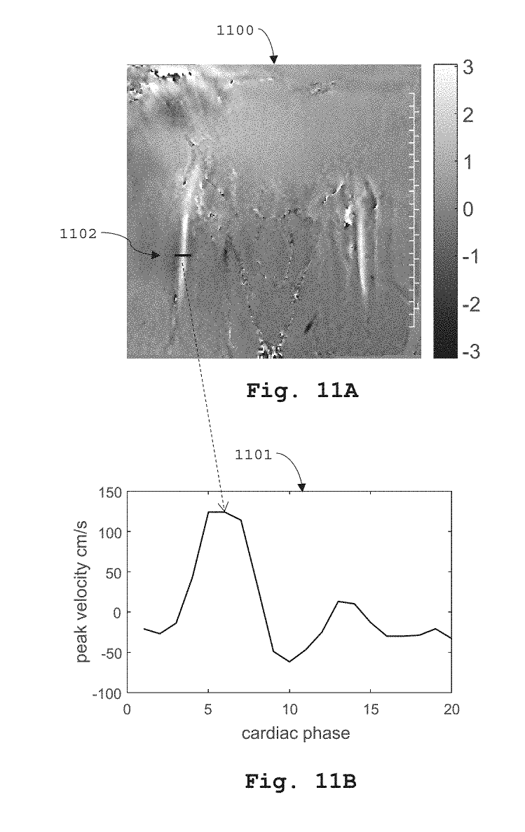

FIG. 10A,B and FIG. 11A,B illustrate exemplary experimental results of using the co-imaging/processing techniques described herein. MRE data were collected from the femoral artery of a healthy volunteer using a three Tesla MRI scanner (Siemens Healthcare, Erlangen). A mechanical driver was used to create a 160 Hz oscillatory signal and was placed on the subject's upper thigh to create corresponding shear waves in the subject. Four phase offsets, each with positive and negative polarity of CG, were collected during the acquisition resulting in the four MRE images shown in FIG. 10A. The MRE images in FIG. 10A correspond to four phase images 1000, 1001, 1002, 1003 of the propagation of the mechanical wave through the right femoral artery (i.e., snap-shots of the wave as it propagates). Using the four phase images 1000, 1001, 1002, 1003, a corresponding tissue stiffness map 1004 (shown in FIG. 10B) was created by estimating the local spatial frequency of the waves. FIGS. 11A, 11B illustrate the results of the velocity imaging. A velocity map 1100 is shown in FIG. 11A, while a corresponding peak-velocity profile 1101 calculated from the map is shown in FIG. 11B. The peak-velocity profile corresponds to a small one-dimensional segment 1102 (shown as a line) across the right femoral artery shown in FIG. 11A.

As compared to conventional flow imaging, the techniques described here (i.e., “elastoflow” imaging) may be used to obtain images with improved (i.e., increased) signal-to-noise ratios.

In the specification and/or figures, typical embodiments of the invention have been disclosed. The present invention is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

1. A method for simultaneously encoding oscillatory tissue motion and fluid flow in a magnetic resonance imaging (MRI) acquisition, the method comprising:
applying an oscillatory motion to tissues located within an MRI field of view, the oscillatory motion creating shear waves in the tissues;
applying a motion encoding gradient (MEG) waveform to encode the shear waves into spin phase;
applying a velocity encoding gradient (VEG) waveform to encode fluid flow into spin phase;
creating a combined gradient waveform that is a weighted combination of the MEG waveform and the VEG waveform; and
applying a combined gradient (CG) waveform to one or more gradients in a pulse sequence for the MRI acquisition.

2. The method according to claim 1, wherein the pulse sequence is a spin-echo (SE) based pulse sequence or a gradient-recalled echo (GRE) based pulse sequence.

3. The method according to claim 2, wherein the CG waveform is applied to the one or more gradients in the pulse sequence after a 90 degree radio-frequency (RF) pulse and before a readout gradient.

4. The method according to claim 1, wherein the one or more gradients in the pulse sequence are aligned with a direction of the shear wave's propagation and/or the direction of the fluid flow.

5. The method according to claim 1, wherein MEG waveform is a waveform in which an inner product between the waveform and the oscillatory motion is nonzero.

6. The method according to claim 5, wherein the waveform is a W₁₋₂₋₁ waveform used in magnetic resonance elastography (MRE).

7. The method according to claim 6, wherein a frequency of the W₁₋₂₋₁ waveform is adjusted to match frequency of the oscillatory motion.

8. The method according to claim 6, wherein the oscillatory motion is adjusted to have a particular phase offset with the W₁₋₂₋₁ waveform.

9. The method according to claim 1, wherein the VEG waveform is a waveform with a non-zero first moment.

10. The method according to claim 9, wherein the VEG waveform is a W₁₋₂₋₁ waveform used in magnetic resonance velocity imaging.

11. The method according to claim 1, wherein the creating a CG waveform that is the weighted combination of the MEG waveform and the VEG waveform, comprises calculating a combined waveform from an equation:
CG=(1−k₁)×W₁₋₂₋₁+k₁×W₁₋₁,
wherein CG is the combined waveform, W₁₋₂₋₁ is a repeating-bipolar waveform used in magnetic resonance elastography (MRE), W₁₋₁ is a nonrepeating-bipolar waveform used in magnetic resonance velocity imaging, and k₁ is a constant that is adjustable from zero to one.

12. The method according to claim 11, wherein k₁ is adjusted so that, during the MRI acquisition, an accumulation of spin phase resulting from tissue displacement and an accumulation of spin phase resulting from fluid-flow velocity are approximately equal.

13. The method according to claim 1, further comprising:
phase shifting the applied oscillatory motion to create a new phase offset between the MEG waveform and the oscillatory motion;
applying the CG waveform to one or more gradients in a pulse sequence for a subsequent MRI acquisition at the new phase offset; and
repeating the steps of phase shifting and applying to sample the shear waves as they propagate through the tissues.

14. A method for obtaining, simultaneously, a tissue stiffness map and a blood flow map from an MRI scan of a subject, the method comprising:
obtaining a pair of complex-valued images, wherein the obtaining comprises:
applying an oscillatory motion to the subject, the oscillatory motion creating shear waves in the subject;
applying a first combined gradient (CG) waveform to one or more gradients in a pulse sequence for the MRI scan;
obtaining a first complex-valued image;
applying a second CG, wherein the second CG has a polarity opposite to the first CG;
obtaining a second complex-valued image, wherein the first complex-valued image and the second complex-valued image form an image pair;
adjusting a phase offset between the CG waveform and the oscillatory motion; and
obtain other image pairs by repeating the steps of obtaining pairs of complex-valued images and adjusting the phase offset;
creating a phase maps for each image pair by multiplying the first complex-value image with a complex conjugate of the second complex-value image, wherein each phase map's background phase is cancelled;
transforming, using a Fourier transform, the phase maps along an offset direction to produce (i) flow information grouped around a baseband and (ii) oscillatory motion information grouped around a first harmonic of a frequency of the oscillatory motion;
separating first-harmonic information from the baseband information;
using the first-harmonic information to create a map of tissue stiffness in the subject; and
using the baseband information to create a map of blood flow in the subject.

15. The method according to claim 14, wherein the CG waveform is a weighted combination of a motion encoding gradient (MEG) waveform and a velocity encoding gradient (VEG) waveform.

16. The method according to claim 15, wherein the weighted combination is calculated using:
CG=(1−k₁)×(MEG)+(k₁)×(VEG),
wherein k₁ is a constant that is adjustable from zero to one.

17. The method according to claim 16, wherein k₁ is determined as a result of a preliminary MRI scan.

18. The method according to claim 15, wherein the motion encoding gradient (MEG) waveform and the oscillatory motion have equal frequencies in a range of 50-500 Hertz (Hz).

19. The method according to claim 15, wherein the motion encoding gradient (MEG) waveform and the oscillatory motion have different frequencies in a range of 50-500 Hertz (Hz).

20. The method according to claim 14, wherein the obtaining, simultaneously, a tissue stiffness map and a blood flow map from an MRI scan requires less time than obtaining, separately a tissue stiffness map from a first MRI scan and a blood flow map from a second MRI scan.
